(19) United States
(12) Patent Application Publication
(10) Pub. No.: US 2023/0001138 A1
(43) Pub. Date: Jan. 5, 2023

(54) SEPTAL CROSSING SYSTEM

(71) Applicant: TAU-PNU MEDICAL CO., LTD, Busan (KR)

(72) Inventor: Jung-Hong Kim, Busan (KR)

(21) Appl. No.: 17/783,605

(22) PCT Filed: Dec. 14, 2020
(86) PCT No.: PCT/US2020/064964

§ 371 Date: Jun. 8, 2022

Related U.S. Application Data
(60) Provisional application No. 62/948,197, filed on Dec. 13, 2019.
(60) Provisional application No. 63/125,346, filed on Dec. 14, 2020.

(51) Int. Cl. A61M 25/00; A61M 25/01

(52) U.S. Cl. CPC A61M 25/0029; A61M 25/0136; A61M 25/0138; A61M 25/0147

(57) ABSTRACT

A septal cross system is provided for a cerclage procedure for treating dysfunctional heart. The cerclage septal cross system includes a puncture catheter and a capture catheter. The puncture catheter a puncture catheter comprises a first lumen for a guidewire to be inserted thereinto. A coil element is arranged in the distal portion of the puncture catheter. The distal end of the pull-wire is attached to the distal portion of the coil element. The proximal end of the pull-wire is extended to the distal portion of the puncture catheter. The pull-wire is configured to bend inwardly the distal portion of the puncture catheter. A capture catheter comprises a first lumen for a first guidewire to be inserted thereinto and a second lumen for a second guidewire to be inserted thereinto. The distal end of the second wire has a snare wherein the distal portion is deflectable.

### RELATED APPLICATIONS

This application claims the benefit of U.S. priorities of U.S. Provisional Patent Application No. 62/948,197 (filed on Dec. 13, 2019) and Application No. 63/125,346 (filed on Dec. 14, 2020), the subject matter of which are hereby incorporated by reference in its entirety.

Present invention generally relates to a procedure for treating heart valvular insufficiency, and more specifically to a septal cross system, and a method for using a target capture catheter and a septal puncture catheter in the procedure.

### BACKGROUND

In a cerclage procedure for treating valvular insufficiencies, there is a need to accurately locate the Right Ventricular Outflow Track (RVOT). However, in the fluoroscopic view of the procedure, it not possible to accurately locate the RVOT. Second, even if the RVOT is accurately found, there is a need to stabilize the catheter at RVOT and maintain its position without moving throughout the heart contractions. Lastly, since there are patients who do not have the septal vein, there is a need to accurately puncture through the IVS directly from the coronary sinus. Further, since sometimes it is difficult to access the septal vein with a guidewire, there is a need for a method and a system to accurately puncture through the IVS into the RVOT directly from the coronary sinus without using the septal vein as a pathway.

### SUMMARY

According to one aspect of the present invention, a puncture catheter comprises a first lumen for a guidewire to be inserted thereinto. A coil element is arranged in the distal portion of the puncture catheter. The distal end of the pull-wire is attached to the distal portion of the coil element. The proximal end of the pull-wire is extended to the distal portion of the puncture catheter. The pull-wire is configured to bend the distal portion of the puncture catheter inwardly so as to have an angled configuration.

According to one aspect of the present invention, a capture catheter comprises a first lumen for a first guidewire to be inserted thereinto and a second lumen for a second guidewire to be inserted thereinto. The distal end of the second wire has a snare wherein the distal portion has a deflectable tip.

According to one aspect of the present invention, a cerclage septal cross system comprises the puncture catheter and the capture catheter.

### BRIEF DESCRIPTION OF THE DRAWINGS

FIG. 1 shows the puncture device.

FIG. 2 shows the distal portion of the puncture device.

FIG. 3 shows the distal portion of the puncture device.

FIG. 4 shows the side view of the puncture device.

FIG. 5 shows the side view of the puncture device.

FIG. 6 shows the side view of the puncture device.

FIG. 7 shows the bendable part of the puncture device.

FIG. 8 shows the bendable part of the puncture device.

FIG. 9 shows the puncture device in the coronary vein.

FIG. 10 shows the puncture device in the coronary vein.

FIG. 11 shows the puncture device in the coronary vein.

FIG. 12 shows the puncture device puncturing the target site.

FIG. 13 shows the puncture device under the imaging analysis.

FIG. 14 shows the puncture device in operation.

FIG. 15 shows the puncture device in operation.

FIG. 16 shows the puncture device in operation.

FIG. 17 shows a cross-sectional view of the puncture device.

FIG. 18 shows a cross-sectional view of the puncture device.

FIG. 19 shows a cross-sectional view of the puncture device and lumens.

FIG. 20 shows the capture device.

FIG. 21 shows the hub of the capture device.

FIG. 22 shows the top and side view of the capture device.

FIG. 23 shows the cross-sectional view of the capture device.

FIG. 24 shows the capture device positioned at the target site.

FIG. 25 shows the target site of the RVOT septum.

FIG. 26 shows the capture device positioned at the RVOT septum.

FIG. 27 shows the capture device captures the puncture wire.

FIG. 28 shows the puncture wire passing through the snare of the capture catheter.

FIG. 29 shows an actual image of the stabilizing wire and the target snare.

FIG. 30 shows an overview of the septal cross system.

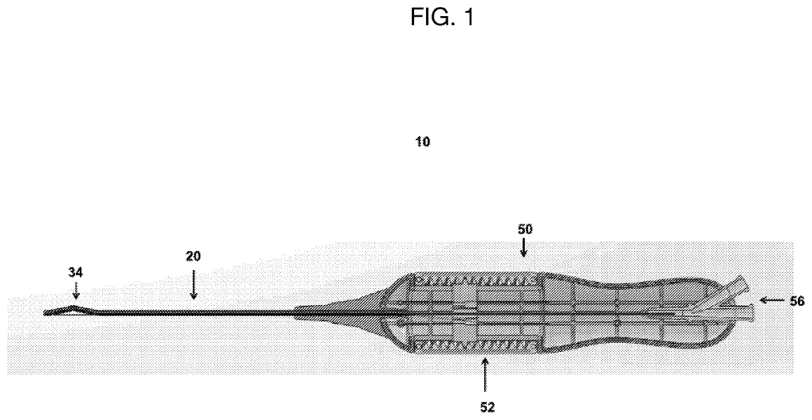

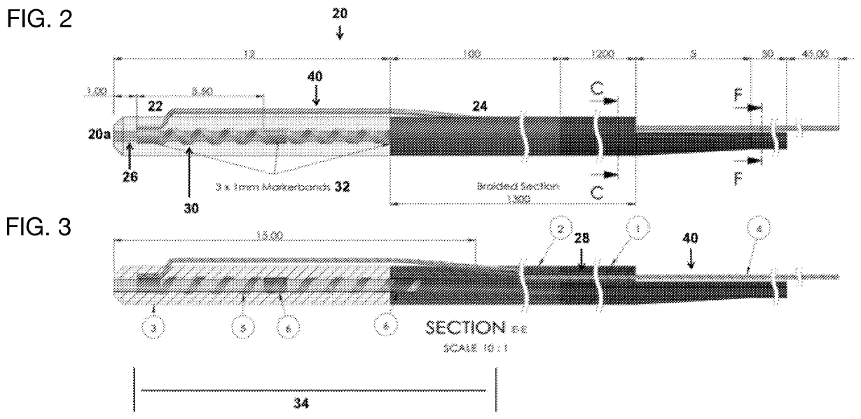

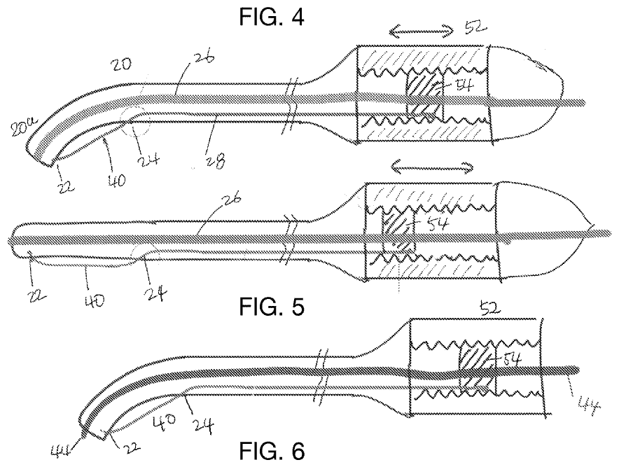

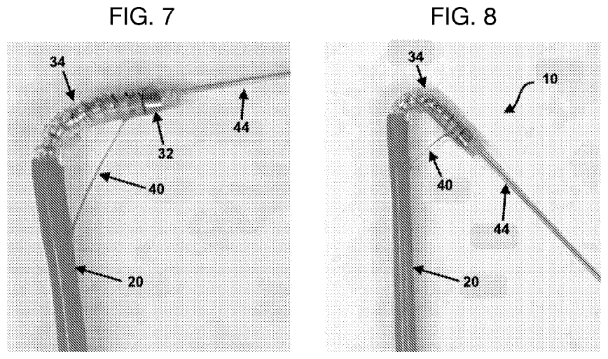

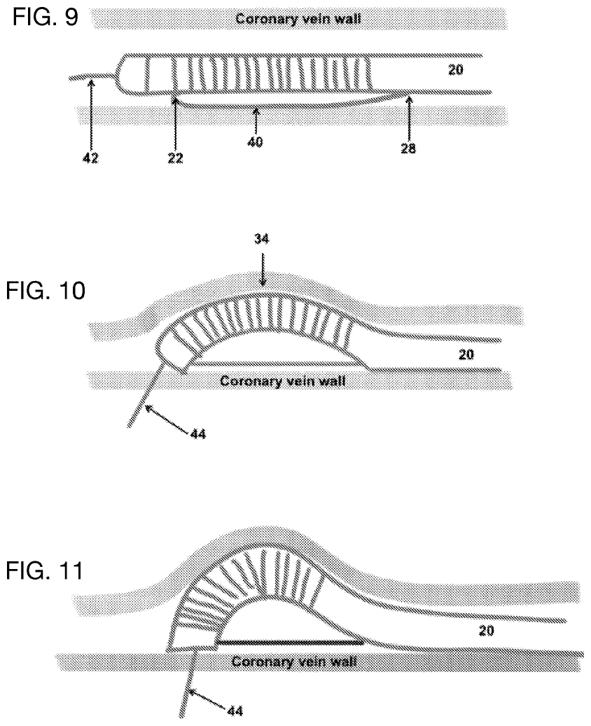

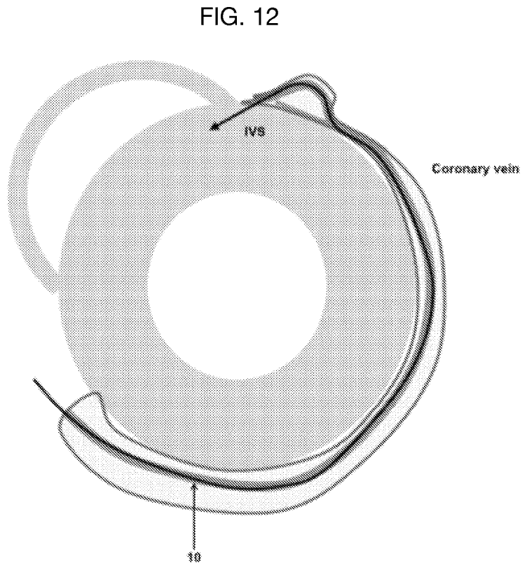

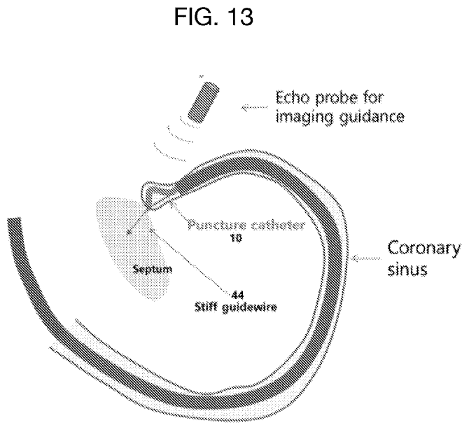

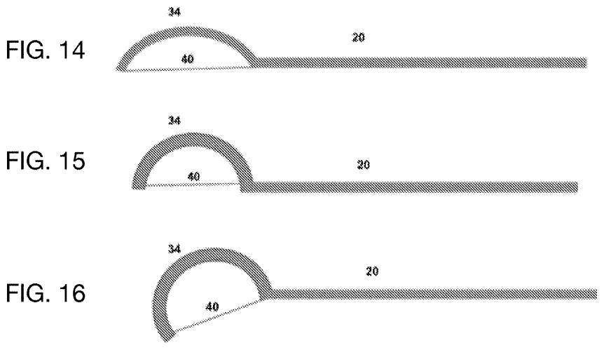

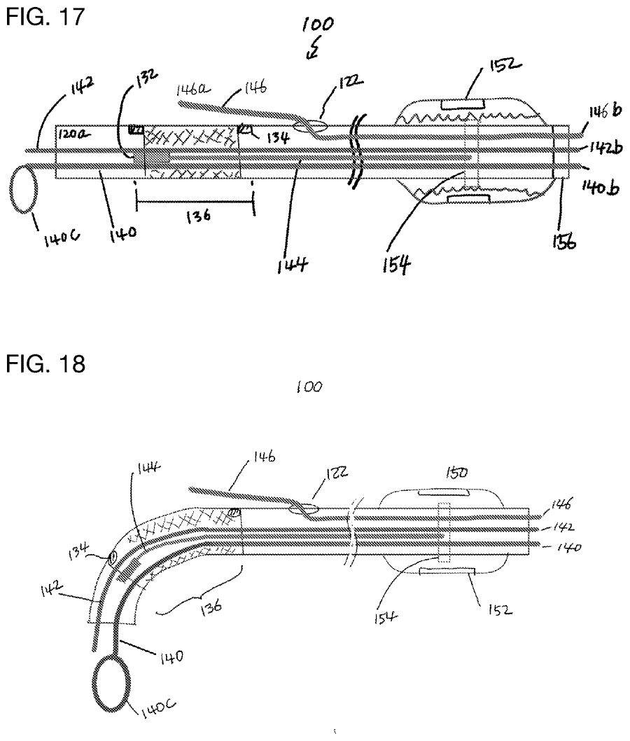

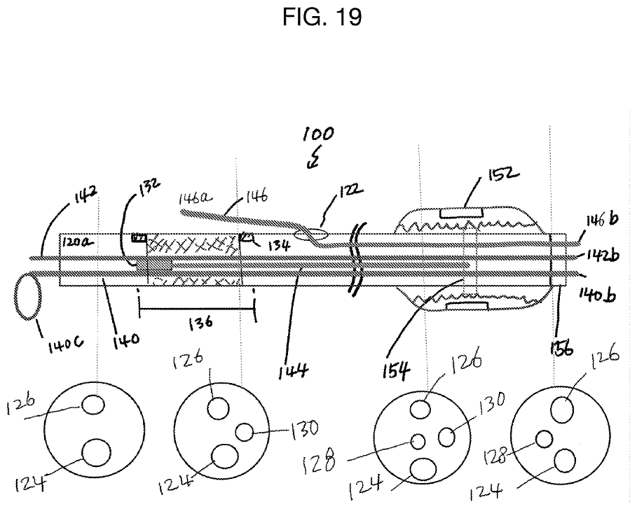

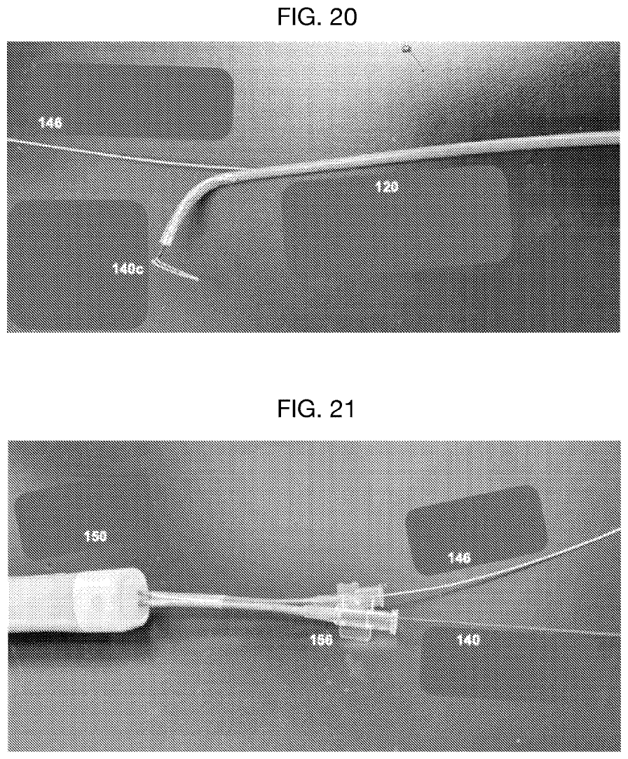

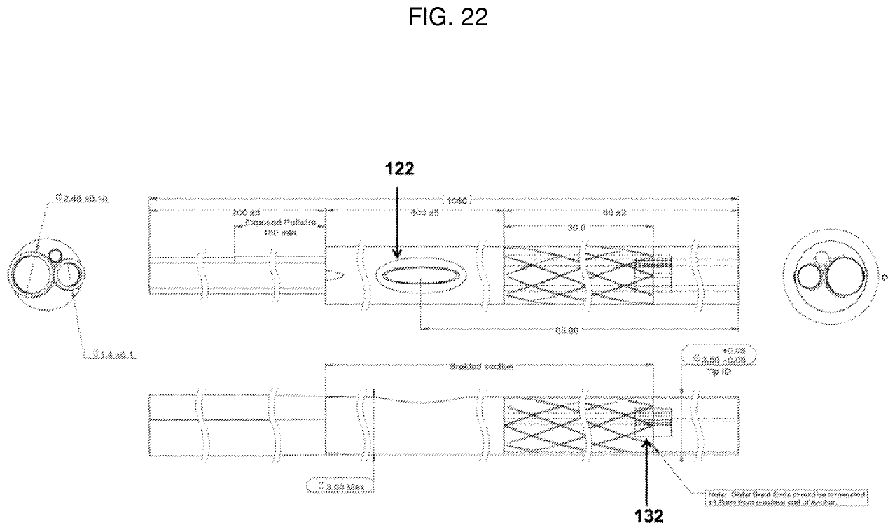

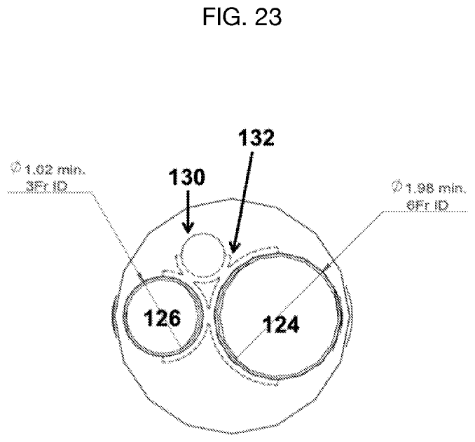

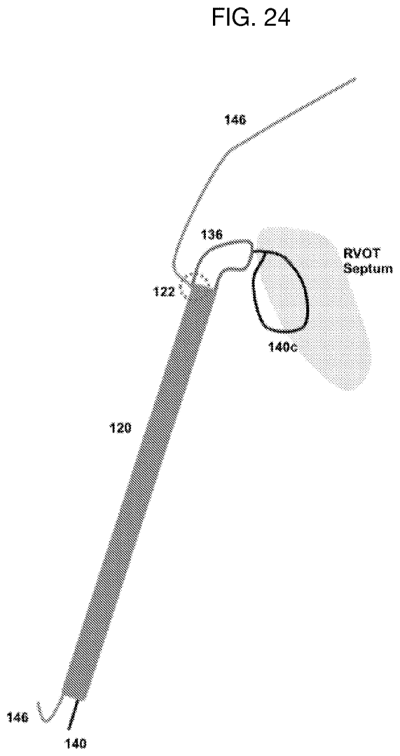

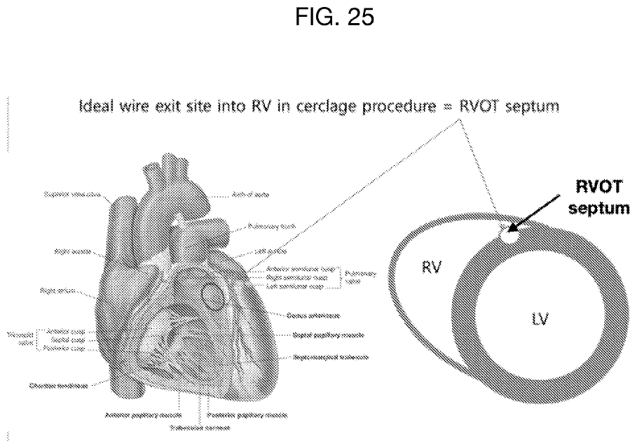

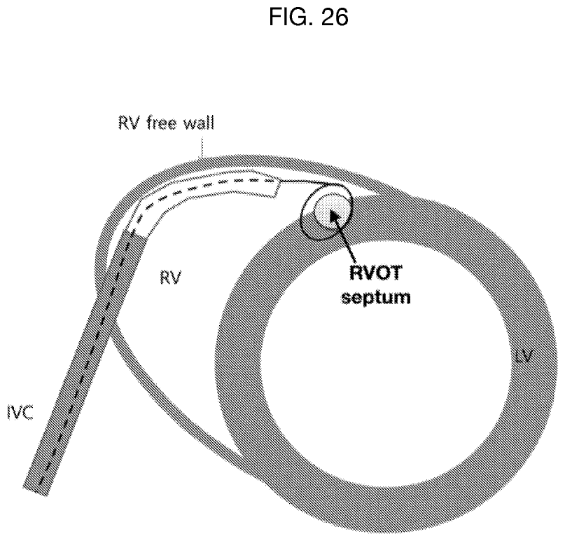

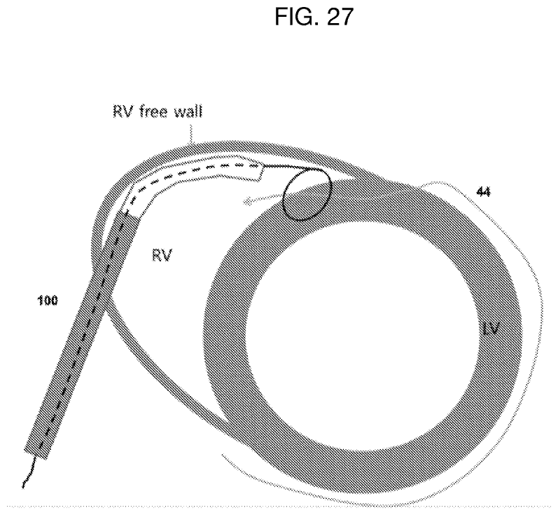

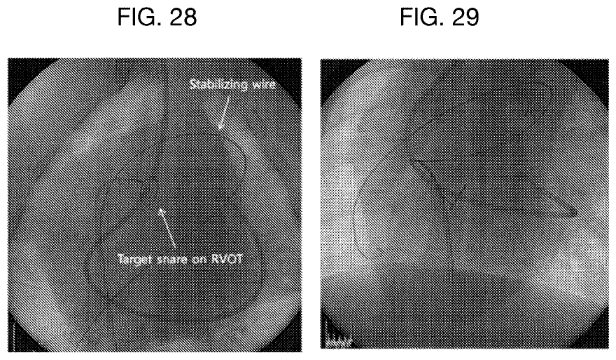

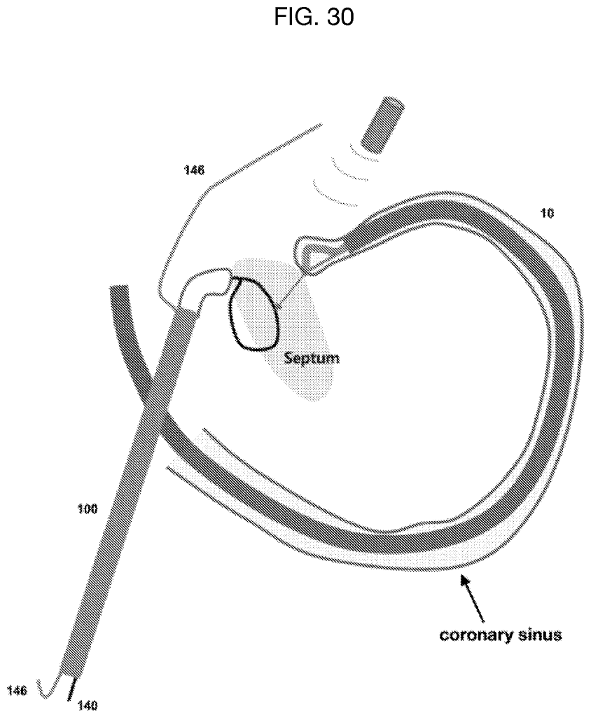

### DETAILED DESCRIPTION OF EMBODIMENTS

### Bendable Puncture Device

A bendable puncture device 10 in an embodiment of the present invention will be described with reference to the drawings. The bendable puncture device 10 of the present embodiment is a tubular medical instrument to be used while being inserted into a patient's body.

FIG. shows the puncture device for the septal crossing system. The bendable puncture device 10 includes a flexible tubular multi-lumen tube (main tube) 20 includes a distal end 20a, a proximal end 20b, and a bendable part 22 provided at the distal end 20a of the main tube 20 and capable of being bent, and a steering part 30 fixed to the proximal end 20b of the main tube 20 for steering the bendable part 22.

### Main Tube 20

The main tube 20 is a flexible multi-lumen tube to be inserted into the coronary sinus. The main tube 20 has a distal end 20a and a proximal end 20b. The main tube 20 includes a first lumen 26. The first lumen 26 has a distal end 26a and a proximal end 26b that are open to each of the distal end 20a and proximal end 20b of the main tube 20. The proximal end 20b of the main tube 20 is fixed to the steering part 50. The first lumen 26 is configured for insertion therethrough of the guide wire 42 or the puncture wire 44.

### First and Second Side Holes 22 & 24

The main tube 20 further includes a first side hole 22 and a second side hole 24. Each of the first and second side holes 22 and 24 are formed on the distal end portion of the main tube 20. The main tube 20 further includes a second lumen 28. The second lumen 28 has a distal end 28a and a proximal end 28b. The distal end 28a of the second lumen 28 is open to the second side hole 24. The first and second side holes 22 and 24 are configured for insertion therethrough of the steering wire.

### Second Lumen 28

The main tube 20 further includes a second lumen 28. The second lumen 28 has a distal end 28a and a proximal end 28b. The distal end 28a of the second lumen 28 is open to the second side hole 24. The proximal end 28b of the second lumen is extended to the steering part 50. The second lumen 28 is configured for insertion therethrough of the steering wire 40.

### Steering Wire 40

The main tube 20 uses a steering wire 40 for bending the distal end 20a of the main tube 20. The steering wire 40 has a distal end 40a and a proximal end 40b. The distal end 40a is attached to the distal end 20a of the main tube 20. The proximal end 40b is attached to the steering part 50. The steering wire 40 is exposed over the portion defined between the first side hole 22 and the second side hole 24 as shown in FIG.

### Coil Element 30

The main tube 20 further includes a coil element 30 in support of the puncturing position. The coil element 30 has a distal end 30a and a proximal end 30b. The distal end 30a is provided proximate the first side hole 22 of the main tube 20, while the proximal end 30b is provided proximate the second side hole 24 of the main tube 20. The coil element 30 is configured to encircle the first lumen 26 in a spiral shape for reinforcing the first lumen 26. The coil element 30 and the first lumen 26 have such a common axis that the coil element 30 bends coaxially as the main tube 20 is being bent in support of the puncturing position.

### Marker Band 32

The main tube 20 further includes at least a marker band 32 for imaging analysis. As shown in FIG. the distal end 20a of the main tube 20 is provided with marker bands 32 made of a material appearing on an X-ray transparent image. The multiple marker bands 32 allow the operator to confirm the amount of bending degree of the main tube 20 before puncturing with reference to the X-ray transparent image.

### Bendable Part 34

A bendable part 34 is defined in the main tube 20 configured for being bent.

The bendable part 34 has a distal end 34a and a proximal end 34b. In one example of the embodiment, the bendable part 34 is preferably arranged between the first side hole 22 and the second side hole 24 of the main tube 20. It should be appreciated such arrangement can be varied depending upon the patient anatomy. As shown in FIG. the steering wire 40 is preferably exposed over the portion defined between the first side hole 22 and the second side hole 24.

### A Set of Wires for Operation

The main tube 20 uses a set of wires for operation. The first lumen 26 is configured for insertion therethrough of the guide wire 42. The guide wire 42 is exchanged with the puncture wire 44 when the main tube 20 is positioned at the target site. The second lumen 28 is configured for insertion therethrough of the steering wire 44. The distal end 40a of the steering wire 44 is attached to the distal end 20a of the main tube 20 and exits through the first hole 22. The steering wire 44 then return through the second hole 24 and extends through the second lumen 26. The proximal end 40b of the steering wire is attached to the steering part 50. Before puncturing, the operator finds an optimal puncturing angle of the bendable part 34 by pulling the puncturing wire 44 from the steering part 50.

### Steering Part 50

The bendable puncture device 10 further includes the steering part 50 for steering the bendable part 34. The steering part 50 has a distal end 50a and a proximal end 50b as shown in FIG. The steering part 50 includes a knob 52, a slider 52, a hub 56 for steering operation. The steering part 50 further a lumen 58 for insertion therethrough of the guide wire 42 or the puncture wire 44 through the hub 56. The lumen 58 is open to the first lumen 26 for communication.

### Knob 52 and Slider 52

The knob 52 operates with the slider 54. The slider 54 moves back or forth by rotating the knob 52 as shown in FIG. The proximal end 40b is attached to the slider 54 in such a way the slider can pull or push the steering wire 40. For example, the operator can adjust the puncturing angle of the bendable part 34 by rotating the knob 42.

### Hub 56

The hub 56 is used for insertion therethrough of the guide wire 42 or the puncture wire 44 during the procedure. The hub 56 is open to the steering part lumen 58 which is also open to the first lumen 26 of the main tube 20. It should be appreciated that various types of the hub can be used depending upon the procedure.

### Capture Device 100

FIG. shows the capture device 100 for the septa crossing system. The capture device 100 includes a flexible tubular multi-lumen tube (main tube) 120. The capture device 100 further includes a steering part 150 for bending the main tube 120.

### Main Tube 120

The main tube 120 is a flexible multi-lumen tube for capturing. The main tube 120 has a distal end 120a and a proximal end 120b. The proximal end 120b of the main tube 120 is fixed to the steering part 150. The first lumen 124 is configured for insertion therethrough of the guide wire (or stabilizing wire) 140.

### Side Hole 122

The main tube 120 includes a side hole 122. The side hole 122 is formed on the main tube 120. The side hole 122 is configured for insertion therethrough of a stabilizing wire 142.

### First Lumen 124 for Snare Wire 140

The main tube 120 further includes a first lumen 124. The first lumen 124 has a distal end 124a and a proximal end 124b that are open to each of the distal end 120a and proximal end 122a of the main tube 120. The first lumen 124 is configured for insertion therethrough of a snare wire 140.

### Second Lumen 126 for Extra Guide Wire 142

The main tube 120 further includes a second lumen 126. The second lumen 126 has a distal end 126a and a proximal end 126b that are open to each of the distal end 120a and proximal end 122a of the main tube 120. The second lumen 126 is configured for insertion therethrough of a guide wire 142

### Third Lumen 128 for Steering Wire 144

The main tube 120 further includes a third lumen 126. The third lumen 128 has a distal end 128a and a proximal end 128b. The third lumen 126 is configured for insertion therethrough of a steering wire 144.

### Fourth Lumen 130 for Stabilizing Wire 146

The main tube 120 further includes a fourth lumen 130. The fourth lumen 130 has a distal end 130a and a proximal end 130b. The fourth lumen 126 is configured for insertion therethrough of the stabilizing wire 146. The distal end 130a of the fourth lumen 130 is open to the side hole 122 of the main tube 120.

### Steering Anchor 132

The main tube 120 further includes a steering anchor 132 for bending the main tube 120. The steering anchor 132 is provided in the distal end portion of the main tube 120. As shown in the FIG. the steering anchor 132 has a X shape partially encircling the first lumen 124 and the second lumen 126 longitudinally. The steering anchor 132 includes an anchor connector 132a for attaching the distal end 144a of the steering wire 144.

### Marker Band 134

The main tube 120 further includes at least a marker band 134 for imaging analysis. As shown in FIG. the distal end 120a of the main tube 120 is provided with marker bands 134 made of a material appearing on an X-ray transparent image. The multiple marker bands 134 allow the operator to confirm the amount of bending degree of the main tube 120 before puncturing with reference to the X-ray transparent image.

### Bendable Part 136

The main tube 120 further includes a bendable part 136 defined in the distal portion of the main tube 120. In one example of the embodiment, the bendable portion is made in a braided configuration. The bendable part 136 is capable of being bent by operating the handle part 150 of the puncture device 100.

### Snare Wire 140

The main tube 120 uses a snare wire 140 for capturing a target wire. The snare wire 140 has a distal end 140a and a proximal end 140b. The distal end 140a is attached to a snare 140c as shown in FIG. The proximal end 140b is extended to the handle part 150 through the first lumen 124.

### Guide Wire 142

The main tube 120 further uses a guide wire 142. The guide wire 142 has a distal end 142a and a proximal end 142b. The guide wire 142 extends from the distal end 120a to the proximal end 120b of the main tube 120 through the second lumen 126.

### Steering Wire 144

The main tube 120 further uses a steering wire 142 for bending the main tube 120. The steering wire 144 has a distal end 144a and a proximal end 144b. The distal end 144a is attached to the steering anchor 132 for bending the main tube 120. The proximal end 114b is attached to the hand part 150 for steering.

### Stabilizing Wire 146

The main tube 120 further uses a stabilizing wire 146 for positioning the main tube 120 to a target site. The stabilizing wire 146 has a distal end 146a and a proximal end 146b. The stabilizing wire 146 passes through the side hole 122 and extends to the handle part through the fourth lumen 130.

### Steering Part 150

The capture device 100 includes the steering part 150 for steering the bendable part 136. The steering part 150 has a distal end 150a and a proximal end 150b as shown in FIG. The steering part 150 includes a knob 152, a slider 152, a hub 156 for steering operation. The first lumen 124, the second lumen 126, and the fourth lumen 130 respectively pass through the steering part 150 and connect to the hub 156.

### Knob 152 and Slider 154

The knob 152 operates with the slider 154. The slider 154 moves back or forth by rotating the knob 152 as shown in FIG. The proximal end 140b of the steering wire 144 is attached to the slider 154 in such a way the slider 154 can pull or push the steering wire 144. For example, the operator can adjust the capturing angle of the bendable part 136 by rotating the knob 142.

### Hub 156

The hub 156 of the steering part 150 has a plurality of port. Each port of the hub is used for insertion therethrough of the snare wire 140, the guide wire 142, the stabilizing wire 146 during the procedure.

1. A bendable catheter comprising:
a flexible tubular main tube having a distal end and a proximal end and a first lumen extending therebetween for insertion of a guide wire, the main tube having a first side hole formed on the distal end, the main tube having a second lumen open to the side hole, the main tube having a steering wire, the steering wire having a distal end and a proximal end, the distal end of the steering wire attached on the distal end of the main tube, the steering wire configured to pass over the outer surface of the main tube and enter into the first side hole, the proximal end of the steering wire configure to be pulled or pushed for bending the distal end of the main tube.

2. The bendable catheter according to claim 1, further comprising a coil element provided in the distal end of the main tube.

3. The bendable catheter according to claim 1, further comprising a steering part provided in the proximal end of the main tube.

4. The bendable catheter according to claim 1, further comprising a second side hole formed on the distal end of the main tube.

5. The bendable catheter according to claim 1, further comprising at lease a marker band arranged on the distal end of the main tube.

6. A bendable catheter comprising: a flexible tubular main tube having a distal end and a proximal end and a first lumen extending therebetween for insertion of a guide wire, the main tube having a first side hole formed on the distal end, the main tube having a second lumen open to the side hole, the main tube having a steering wire, the steering wire having a distal end and a proximal end, the main tube having an anchor provided in the distal end of the main tube, the distal end of the steering wire attached on the anchor, the proximal end of the steering wire configured to be pulled or pushed for bending the distal end of the main tube.

7. The bendable catheter according to claim 6, further comprising at lease a marker band arranged on the distal end of the main tube.

8. A septal crossing system comprising:
a puncture catheter comprising a flexible tubular main tube having a distal end and a proximal end and a first lumen extending therebetween for insertion of a guide wire, the main tube having a first side hole formed on the distal end, the main tube having a second lumen open to the side hole, the main tube having a steering wire, the steering wire having a distal end and a proximal end, the distal end of the steering wire attached on the distal end of the main tube, the steering wire configured to pass over the outer surface of the main tube and enter into the first side hole, the proximal end of the steering wire configure to be pulled or pushed for bending the distal end of the main tube; and
a capture catheter comprising: a flexible tubular main tube having a distal end and a proximal end and a first lumen extending therebetween for insertion of a guide wire, the main tube having a first side hole formed on the distal end, the main tube having a second lumen open to the side hole, the main tube having a steering wire, the steering wire having a distal end and a proximal end, the main tube having an anchor provided in the distal end of the main tube, the distal end of the steering wire attached on the anchor, the proximal end of the steering wire configured to be pulled or pushed for bending the distal end of the main tube.
